(19) United States
(12) Patent Application Publication
(10) Pub. No.: US 2022/0273233 A1
(43) Pub. Date: Sep. 1, 2022

(54) Brain Activity Derived Formulation of Target Sleep Routine for a User

(71) Applicant: HI LLC, Los Angeles, CA (US)

(72) Inventors: Bryan Johnson, Culver City, CA (US); Ryan Field, Culver City, CA (US); Katherine Perdue, Los Angeles, CA (US)

(21) Appl. No.: 17/592,615

(22) Filed: Feb. 4, 2022

Related U.S. Application Data
(60) Provisional application No. 63/179,957, filed on Apr. 26, 2021.
(60) Provisional application No. 63/235,039, filed on Aug. 19, 2021.
(60) Provisional application No. 63/173,341, filed on Apr. 9, 2021.
(60) Provisional application No. 63/160,766, filed on Mar. 13, 2021.
(60) Provisional application No. 63/154,123, filed on Feb. 26, 2021.

(51) Int. Cl. A61B 5/00; A61B 5/291; A61B 5/31; A61B 5/384

(52) U.S. Cl. CPC A61B 5/4815; A61B 5/4064; A61B 5/0066; A61B 5/7267; A61B 5/291; A61B 5/31; A61B 5/384; A61B 5/6802; A61B 5/74

(57) ABSTRACT

An illustrative system includes a brain interface system configured to be worn by a user and to output brain activity data associated with the user; a sleep tracking device configured to be worn by the user and to output sleep tracking data associated with the user; and a computing device configured to generate, based on the brain activity data and the sleep tracking data, sleep routine data representative of a target sleep routine for the user.

### RELATED APPLICATIONS

The present application claims priority under 35 U.S.C. § 119(e) to U.S. Provisional Patent Application No. 63/179,957, filed on Apr. 26, 2021, and to U.S. Provisional Patent Application No. 63/235,039, filed on Aug. 19, 2021, and to U.S. Provisional Patent Application No. 63/173,341, filed on Apr. 9, 2021, and to U.S. Provisional Patent Application No. 63/160,766, filed on Mar. 13, 2021 and to U.S. Provisional Patent Application No. 63/154,123, filed on Feb. 26, 2021. These applications are incorporated herein by reference in their respective entireties.

### BACKGROUND INFORMATION

Sleep quality is not only important to overall health, but it also directly affects an individual's day-to-day ability to function both physically and mentally. For example, low quality sleep may negatively affect an individual's ability to perform physical tasks, think clearly, exercise impulse control, and/or interact with others.

Sleep quality for a particular individual depends on a number of different factors, including environmental conditions, settings of devices used by the individual, a mental state of the individual, and physiological functions of the individual. Many of these factors can be controlled or influenced by choices and/or specific actions taken by the individual and/or by one or more computing devices throughout the day. Such choices and/or specific actions that may be taken by an individual may be referred to as a sleep routine for the individual. Unfortunately, ideal sleep routines (i.e., sleep routines that produce high quality sleep) vary greatly from individual to individual and may accordingly be difficult to determine and consistently follow.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an exemplary sleep analysis system.

FIGS. 2-4, 5A and 5B show various optical measurement systems that may implement the brain interface system of FIG. 1.

FIGS. 6-7 show various multimodal measurement systems that may implement the brain interface system of FIG. 1.

FIG. 8 shows an exemplary magnetic field measurement system that may implement the brain interface system of FIG. 1.

FIG. 9 illustrates various components that may be included in a sleep tracking device.

FIG. 10 illustrates a configuration in which a computing device is configured to present content associated with a target sleep routine to the user.

FIG. 11 illustrates a configuration in which a computing device is configured to assist a user in adhering to a target sleep routine.

FIG. 12 illustrates a configuration in which a computing device is configured to control one or more settings of a device.

FIG. 13 illustrates a configuration in which a computing device is configured to apply brain activity data to a machine learning model.

FIG. 14 illustrates a configuration in which a sleep routine module is configured to receive characteristic data representative of a desired characteristic for a user.

FIG. 15 illustrates a configuration in which a sleep routine module is configured to receive task data representative of an upcoming task that a user is to perform.

FIG. 16 illustrates an exemplary method.

FIG. 17 illustrates an exemplary computing device.

### DETAILED DESCRIPTION

Brain activity derived formulation of a target sleep routine for a user is described herein. For example, an illustrative system may include a brain interface system configured to be worn by a user and to output brain activity data associated with the user, a sleep tracking device configured to be worn by the user and to output sleep tracking data associated with the user, and a computing device configured to generate, based on the brain activity data and the sleep tracking data, sleep routine data representative of a target sleep routine for the user.

As used herein, a “sleep routine” for a user refers to a set of one or more actions that the user may perform, one or more choices that a user may make, and/or one or more operations that one or more computing devices may execute that may affect the quality of sleep for the user. Various example sleep routines are described herein. A “target sleep routine” for a user refers to a particular sleep routine that may result in the user obtaining a target sleep quality level. Such target sleep quality level may be objective (e.g., in the form of a quantified sleep performance score as measured by a sleep tracking device and/or by a professional) and/or subjective (e.g., the user may obtain a target sleep quality level if the user feels like he or she has had a sufficient amount of quality sleep during a particular time period and/or if a subjective evaluation by a professional indicates that the user has had a sufficient amount of quality sleep to perform certain tasks). As used herein “sleep quality” and “quality of sleep” refers to a quantifiable effectiveness of sleep, and may depend on a total amount of time that the user sleeps during a given time period (e.g., during the night), an amount of time in particular stages of sleep (e.g., wake, light sleep, deep sleep, and REM sleep), an amount of interrupted sleep, a heart rate of the user while the user sleeps and/or tries to sleep, a blood oxygenation level while the user sleeps and/or tries to sleep, a user's movement during sleep, and/or any other sleep-related factor as may serve a particular implementation.

Benefits of the aspects described herein include an ability to discover a target sleep routine for a user that optimizes impulse control, learning ability, creative ability, emotion regulation, ability to connect more deeply with loved ones and others, and/or other desired traits. Benefits further include helping a user to quit a bad habit (e.g., stop smoking, stop wasting time on social media platforms, etc.). Benefits also include improving a relationship between a user and one or more other people. For example, the aspects described herein may be used to determine a correct sequence leading up to bedtime that minimizes snoring by the user. This may lead to a relationship benefit for those who disturb their partners with snoring. Other benefits will be made apparent herein.

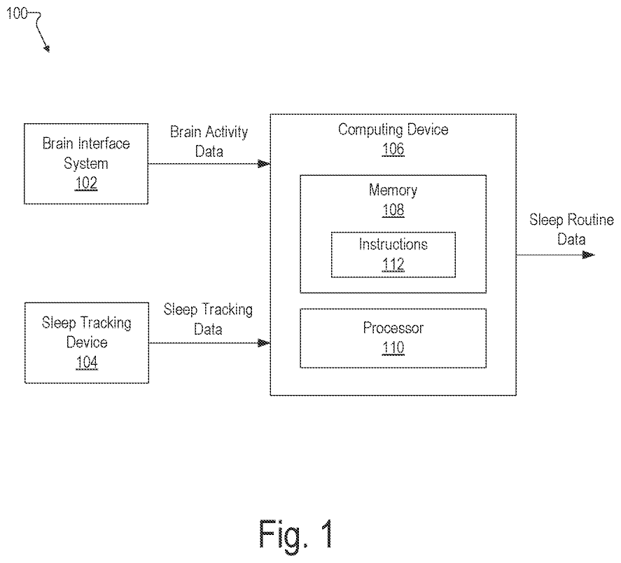

FIG. 1 shows an exemplary sleep analysis system 100. As shown, sleep analysis system 100 includes a brain interface system 102 and a sleep tracking device 104 each configured to be communicatively coupled to a computing device 106.

Brain interface system 102 is configured to output brain activity data associated with a user. As described herein, the brain activity data may include any data output by any of the implementations of brain interface system 102 described herein. For example, the brain activity data may include or be based on optical-based, electrical-based, and/or magnetic field-based measurements of activity within the brain, as described herein. In some examples, the brain activity data may indicate how well the user is able to function mentally during a certain time period (e.g., while the user is awake). For example, the brain activity data may indicate how well the user is able to focus on certain tasks, how well the user is able to exercise impulse control when presented with various temptations and/or choices, what the user's mental state is (e.g., how stressed and/or happy the user is), how well the user gets along with others, etc. In some examples, one or more of these measures may be represented by a single brain activity score that is derived from the brain activity data. This single brain activity score may be generated in any suitable manner.

The measured brain activity could be related to physiological brain states and/or mental brain states, e.g., joy, excitement, relaxation, surprise, fear, stress, anxiety, sadness, anger, disgust, contempt, contentment, calmness, approval, focus, attention, creativity, cognitive assessment, positive or negative reflections/attitude on experiences or the use of objects, etc. Further details on the methods and systems related to a predicted brain state, behavior, preferences, or attitude of the user, and the creation, training, and use of neuromes can be found in U.S. patent application Ser. No. 17/188,298, filed Mar. 1, 2021, issued as U.S. Pat. No. 11,132,625. Exemplary measurement systems and methods using biofeedback for awareness and modulation of mental state are described in more detail in U.S. patent application Ser. No. 16/364,338, filed Mar. 26, 2019, issued as U.S. Pat. No. 11,006,876. Exemplary measurement systems and methods used for detecting and modulating the mental state of a user using entertainment selections, e.g., music, film/video, are described in more detail in U.S. patent application Ser. No. 16/835,972, filed Mar. 31, 2020, issued as U.S. Pat. No. 11,006,878. Exemplary measurement systems and methods used for detecting and modulating the mental state of a user using product formulation from, e.g., beverages, food, selective food/drink ingredients, fragrances, and assessment based on product-elicited brain state measurements are described in more detail in U.S. patent application Ser. No. 16/853,614, filed Apr. 20, 2020, issued as U.S. Pat. No. 11,172,869. Exemplary measurement systems and methods used for detecting and modulating the mental state of a user through awareness of priming effects are described in more detail in U.S. patent application Ser. No. 16/885,596, filed May 28, 2020, published as US2020/0390358A1. Exemplary measurement systems and methods used for wellness therapy, such as pain management regime, are described more fully in U.S. Provisional Application No. 63/188,783, filed May 14, 2021. These applications and corresponding U.S. patents and publications are incorporated herein by reference in their entirety.

Sleep tracking device 104 is configured to output sleep tracking data associated with the user. Sleep tracking data may be representative of any type of sleep tracking measurements performed by sleep tracking device 104, as described herein. For example, sleep tracking data may be representative of a time that the user goes to bed and/or sleep, a time that the user wakes up, a total amount of time that the user sleeps during a given time period (e.g., during the night), an amount of time in particular stages of sleep (e.g., wake, light sleep, deep sleep, and rapid eye movement (REM) sleep), an amount of interrupted sleep, a heart rate of the user while the user sleeps and/or attempts to go to sleep, a blood oxygenation level of the user while the user sleeps and/or attempts to go to sleep, a body temperature of the user while the user sleeps and/or attempts to go to sleep, one or more environmental conditions (e.g., room temperature, room noise, room light, etc.) associated with an environment in which the user sleeps and/or attempts to go to sleep, and/or any other sleep-related measurement as may serve a particular implementation. In some examples, one or more of these sleep-related measurements may be quantified by a single sleep performance score output by sleep tracking device 104 and/or derived from the sleep tracking data output by sleep tracking device 104. This single sleep performance score may be generated in any suitable manner.

Computing device 106 is configured to receive the brain activity data and the sleep tracking data, and, based on the brain activity data and the sleep tracking data, generate sleep routine data. The sleep routine data may be representative of a target sleep routine for the user and may be generated in any suitable manner, examples of which are described herein.

Computing device 106 may be further configured, in some examples, to perform one or more operations based on the sleep tracking data. Example operations are described herein.

Computing device 106 may be implemented by one or more computing devices, such as one or more personal computers, mobile devices (e.g., a mobile phone, a tablet computer, etc.), servers, and/or any other type of computing device as may serve a particular implementation. In some examples, computing device 106 may be configured to be worn by the user at the same time that brain interface system 102 and sleep tracking device 104 are being worn by the user. Alternatively, computing device 106 may not be worn by the user.

As shown, computing device 106 may include memory 108 and a processor 110. Computing device 106 may include additional or alternative components as may serve a particular implementation. Each component may be implemented by any suitable combination of hardware and/or software.

Memory 108 may maintain (e.g., store) executable data used by processor 110 to perform one or more of the operations described herein as being performed by computing device 106. For example, memory 108 may store instructions 112 that may be executed by processor 110 to generate sleep routine data and/or perform one or more operations based on the sleep routine data. Instructions 112 may be implemented by any suitable application, program, software, code, and/or other executable data instance. Memory 108 may also maintain any data received, generated, managed, used, and/or transmitted by processor 110.

Processor 110 may be configured to perform (e.g., execute instructions 112 stored in memory 108 to perform) various operations described herein as being performed by computing device 106. Examples of such operations are described herein.

Brain interface system 102 may be implemented by any suitable wearable brain non-invasive interface system as may serve a particular implementation. For example, brain interface system 102 may be implemented by a wearable optical measurement system configured to perform optical-based brain data acquisition operations, such as any of the wearable optical measurement systems described in U.S. patent application Ser. No. 17/176,315, filed Feb. 16, 2021 and published as US2021/0259638A1; U.S. patent application Ser. No. 17/176,309, filed Feb. 16, 2021 and published as US2021/0259614A1; U.S. patent application Ser. No. 17/176,460, filed Feb. 16, 2021 and issued as U.S. Pat. No. 11,096,620; U.S. patent application Ser. No. 17/176,470, filed Feb. 16, 2021 and published as US2021/0259619A1; U.S. patent application Ser. No. 17/176,487, filed Feb. 16, 2021 and published as US2021/0259632A1; U.S. patent application Ser. No. 17/176,539, filed Feb. 16, 2021 and published as US2021/0259620A1; U.S. patent application Ser. No. 17/176,560, filed Feb. 16, 2021 and published as US2021/0259597A1; U.S. patent application Ser. No. 17/176,466, filed Feb. 16, 2021 and published as US2021/0263320A1; Han Y. Ban, et al., “Kernel Flow; A High Channel Count Scalable TD-fNIRS System,” SPIE Photonics West Conference (Mar. 6, 2021); and Han Y. Ban. et al., “Kernel Flow: a high channel count scalable time-domain functional near-infrared spectroscopy system,” Journal of Biomedical Optics (Jan. 18, 2022), which applications and publications are incorporated herein by reference in their entirety.

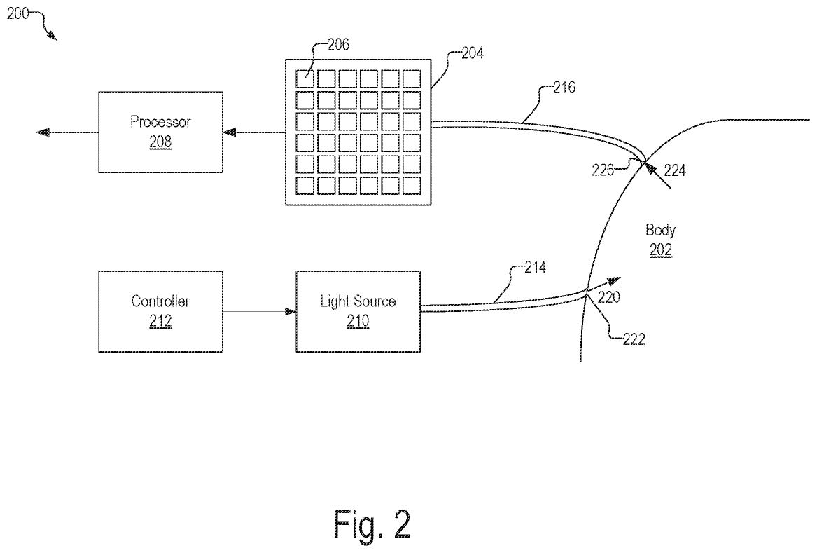

To illustrate, FIGS. 2-4, 5A and 5B, show various optical measurement systems and related components that may implement brain interface system 102. The optical measurement systems described herein are merely illustrative of the many different optical-based brain interface systems that may be used in accordance with the systems and methods described herein.

FIG. 2 shows an optical measurement system 200 that may be configured to perform an optical measurement operation with respect to a body 202 (e.g., the brain). Optical measurement system 200 may, in some examples, be portable and/or wearable by a user.

In some examples, optical measurement operations performed by optical measurement system 200 are associated with a time domain-based optical measurement technique. Example time domain-based optical measurement techniques include, but are not limited to, time-correlated single-photon counting (TCSPC), time domain near infrared spectroscopy (TD-NIRS), time domain diffusive correlation spectroscopy (TD-DCS), and time domain digital optical tomography (TD-DOT).

Optical measurement system 200 (e.g., an optical measurement system that is implemented by a wearable device or other configuration, and that employs a time domain-based (e.g., TD-NIRS) measurement technique) may detect blood oxygenation levels and/or blood volume levels by measuring the change in shape of laser pulses after they have passed through target tissue, e.g., brain, muscle, finger, etc. As used herein, a shape of laser pulses refers to a temporal shape, as represented for example by a histogram generated by a time-to-digital converter (TDC) coupled to an output of a photodetector, as will be described more fully below.

As shown, optical measurement system 200 includes a detector 204 that includes a plurality of individual photodetectors (e.g., photodetector 206), a processor 208 coupled to detector 204, a light source 210, a controller 212, and optical conduits 214 and 216 (e.g., light pipes). However, one or more of these components may not, in certain embodiments, be considered to be a part of optical measurement system 200. For example, in implementations where optical measurement system 200 is wearable by a user, processor 208 and/or controller 212 may in some embodiments be separate from optical measurement system 200 and not configured to be worn by the user.

Detector 204 may include any number of photodetectors 206 as may serve a particular implementation, such as 2ⁿ photodetectors (e.g., 256, 512, . . . , 26384, etc.), where n is an integer greater than or equal to one (e.g., 4, 5, 8, 20, 21, 24, etc.). Photodetectors 206 may be arranged in any suitable manner.

Photodetectors 206 may each be implemented by any suitable circuit configured to detect individual photons of light incident upon photodetectors 206. For example, each photodetector 206 may be implemented by a single photon avalanche diode (SPAD) circuit and/or other circuitry as may serve a particular implementation. The SPAD circuit may be gated in any suitable manner or be configured to operate in a free running mode with passive quenching. For example, photodetectors 206 may be configured to operate in a free-running mode such that photodetectors 206 are not actively armed and disarmed (e.g., at the end of each predetermined gated time window). In contrast, while operating in the free-running mode, photodetectors 206 may be configured to reset within a configurable time period after an occurrence of a photon detection event (i.e., after photodetector 206 detects a photon) and immediately begin detecting new photons. However, only photons detected within a desired time window (e.g., during each gated time window) may be included in the histogram that represents a light pulse response of the target (e.g., a temporal point spread function (TPSF)). The terms histogram and TPSF are used interchangeably herein to refer to a light pulse response of a target.

Processor 208 may be implemented by one or more physical processing (e.g., computing) devices. In some examples, processor 208 may execute instructions (e.g., software) configured to perform one or more of the operations described herein.

Light source 210 may be implemented by any suitable component configured to generate and emit light. For example, light source 210 may be implemented by one or more laser diodes, distributed feedback (DFB) lasers, super luminescent diodes (SLDs), light emitting diodes (LEDs), diode-pumped solid-state (DPSS) lasers, super luminescent light emitting diodes (sLEDs), vertical-cavity surface-emitting lasers (VCSELs), titanium sapphire lasers, micro light emitting diodes (mLEDs), and/or any other suitable laser or light source. In some examples, the light emitted by light source 210 is high coherence light (e.g., light that has a coherence length of at least 5 centimeters) at a predetermined center wavelength.

Light source 210 is controlled by controller 212, which may be implemented by any suitable computing device (e.g., processor 208), integrated circuit, and/or combination of hardware and/or software as may serve a particular implementation. In some examples, controller 212 is configured to control light source 210 by turning light source 210 on and off and/or setting an intensity of light generated by light source 210. Controller 212 may be manually operated by a user, or may be programmed to control light source 210 automatically.

Light emitted by light source 210 may travel via an optical conduit 214 (e.g., a light pipe, a single-mode optical fiber, and/or or a multi-mode optical fiber) to body 202 of a subject. Body 202 may include any suitable turbid medium. For example, in some implementations, body 202 is a brain or any other body part of a human or other animal. Alternatively, body 202 may be a non-living object. For illustrative purposes, it will be assumed in the examples provided herein that body 202 is a human brain.

As indicated by arrow 220, the light emitted by light source 210 enters body 202 at a first location 222 on body 202. Accordingly, a distal end of optical conduit 214 may be positioned at (e.g., right above, in physical contact with, or physically attached to) first location 222 (e.g., to a scalp of the subject). In some examples, the light may emerge from optical conduit 214 and spread out to a certain spot size on body 202 to fall under a predetermined safety limit. At least a portion of the light indicated by arrow 220 may be scattered within body 202.

As used herein, “distal” means nearer, along the optical path of the light emitted by light source 210 or the light received by detector 204, to the target (e.g., within body 202) than to light source 210 or detector 204. Thus, the distal end of optical conduit 214 is nearer to body 202 than to light source 210, and the distal end of optical conduit 216 is nearer to body 202 than to detector 204. Additionally, as used herein, “proximal” means nearer, along the optical path of the light emitted by light source 210 or the light received by detector 204, to light source 210 or detector 204 than to body 202. Thus, the proximal end of optical conduit 214 is nearer to light source 210 than to body 202, and the proximal end of optical conduit 216 is nearer to detector 204 than to body 202.

As shown, the distal end of optical conduit 216 (e.g., a light pipe, a light guide, a waveguide, a single-mode optical fiber, and/or a multi-mode optical fiber) is positioned at (e.g., right above, in physical contact with, or physically attached to) output location 226 on body 202. In this manner, optical conduit 216 may collect at least a portion of the scattered fight (indicated as light 224) as it exits body 202 at location 226 and carry light 224 to detector 204. Light 224 may pass through one or more lenses and/or other optical elements (not shown) that direct light 224 onto each of the photodetectors 206 included in detector 204. In cases where optical conduit 216 is implemented by a light guide, the light guide may be spring loaded and/or have a cantilever mechanism to allow for conformably pressing the light guide firmly against body 202.

Photodetectors 206 may be connected in parallel in detector 204. An output of each of photodetectors 206 may be accumulated to generate an accumulated output of detector 204. Processor 208 may receive the accumulated output and determine, based on the accumulated output, a temporal distribution of photons detected by photodetectors 206. Processor 208 may then generate, based on the temporal distribution, a histogram representing a light pulse response of a target (e.g., brain tissue, blood flow, etc.) in body 202. Such a histogram is illustrative of the various types of brain activity measurements that may be performed by brain interface system 102.

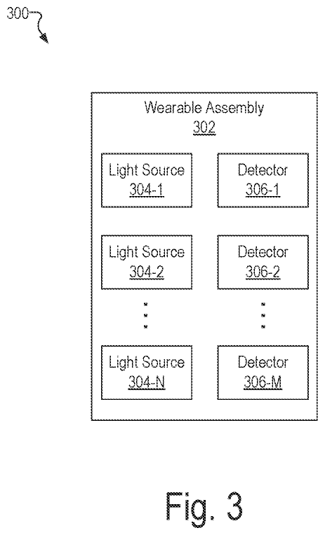

FIG. 3 shows an exemplary optical measurement system 300 in accordance with the principles described herein. Optical measurement system 300 may be an implementation of optical measurement system 200 and, as shown, includes a wearable assembly 302, which includes N light sources 304 (e.g., light sources 304-1 through 304-N) and M detectors 306 (e.g., detectors 306-1 through 306-M). Optical measurement system 300 may include any of the other components of optical measurement system 200 as may serve a particular implementation. N and M may each be any suitable value (i.e., there may be any number of light sources 304 and detectors 306 included in optical measurement system 300 as may serve a particular implementation).

Light sources 304 are each configured to emit light (e.g., a sequence of light pulses) and may be implemented by any of the light sources described herein. Detectors 306 may each be configured to detect arrival times for photons of the light emitted by one or more light sources 304 after the light is scattered by the target. For example, a detector 306 may include a photodetector configured to generate a photodetector output pulse in response to detecting a photon of the light and a time-to-digital converter (TDC) configured to record a timestamp symbol in response to an occurrence of the photodetector output pulse, the timestamp symbol representative of an arrival time for the photon (i.e., when the photon is detected by the photodetector).

Wearable assembly 302 may be implemented by any of the wearable devices, modular assemblies, and/or wearable units described herein. For example, wearable assembly 302 may be implemented by a wearable device (e.g., headgear) configured to be worn on a user's head. Wearable assembly 302 may additionally or alternatively be configured to be worn on any other part of a user's body.

Optical measurement system 300 may be modular in that one or more components of optical measurement system 300 may be removed, changed out, or otherwise modified as may serve a particular implementation. As such, optical measurement system 300 may be configured to conform to three-dimensional surface geometries, such as a user's head. Exemplary modular optical measurement systems comprising a plurality of wearable modules are described in more detail in U.S. patent application Ser. No. 17/176,460, filed Feb. 16, 2021 and issued as U.S. Pat. No. 11,096,620, U.S. patent application Ser. No. 17/176,470, filed Feb. 16, 2021 and published as US2021/0259619A1, U.S. patent application Ser. No. 17/176,487, filed Feb. 16, 2021 and published as US2021/0259632A1, U.S. patent application Ser. No. 17/176,539, filed Feb. 16, 2021 and published as US2021/0259620A1, U.S. patent application Ser. No. 17/176,560, filed Feb. 16, 2021 and published as US2021/0259597A1, and U.S. patent application Ser. No. 17/176,466, filed Feb. 16, 2021 and published as US2021/0263320A1, which applications are incorporated herein by reference in their respective entireties.

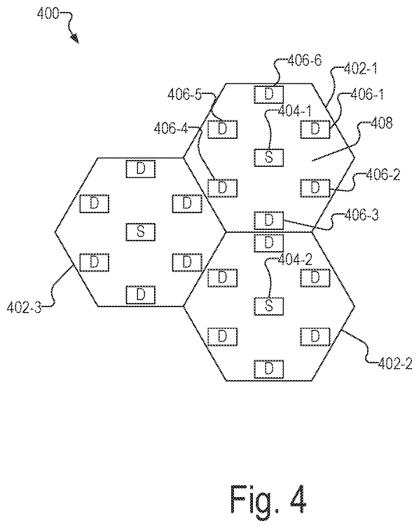

FIG. 4 shows an illustrative modular assembly 400 that may implement optical measurement system 300. Modular assembly 400 is illustrative of the many different implementations of optical measurement system 300 that may be realized in accordance with the principles described herein.

As shown, modular assembly 400 includes a plurality of modules 402 (e.g., modules 402-1 through 402-3) physically distinct one from another. While three modules 402 are shown to be included in modular assembly 400, in alternative configurations, any number of modules 402 (e.g., a single module up to sixteen or more modules) may be included in modular assembly 400.

Each module 402 includes a light source (e.g., light source 404-1 of module 402-1 and light source 404-2 of module 402-2) and a plurality of detectors (e.g., detectors 406-1 through 406-6 of module 402-1). In the particular implementation shown in FIG. 4, each module 402 includes a single light source and six detectors. Each light source is labeled “S” and each detector is labeled “D”.

Each light source depicted in FIG. 4 may be implemented by one or more light sources similar to light source 210 and may be configured to emit light directed at a target (e.g., the brain).

Each light source depicted in FIG. 4 may be located at a center region of a surface of the light source's corresponding module. For example, light source 404-1 is located at a center region of a surface 408 of module 402-1. In alternative implementations, a light source of a module may be located away from a center region of the module.

Each detector depicted in FIG. 4 may implement or be similar to detector 204 and may include a plurality of photodetectors (e.g., SPADs) as well as other circuitry (e.g., TDCs), and may be configured to detect arrival times for photons of the light emitted by one or more light sources after the light is scattered by the target.

The detectors of a module may be distributed around the light source of the module. For example, detectors 406 of module 402-1 are distributed around light source 404-1 on surface 408 of module 402-1. In this configuration, detectors 406 may be configured to detect photon arrival times for photons included in light pulses emitted by light source 404-1. In some examples, one or more detectors 406 may be close enough to other light sources to detect photon arrival times for photons included in light pulses emitted by the other light sources. For example, because detector 406-3 is adjacent to module 402-2, detector 406-3 may be configured to detect photon arrival times for photons included in light pulses emitted by light source 404-2 (in addition to detecting photon arrival times for photons included in light pulses emitted by light source 404-1).

In some examples, the detectors of a module may all be equidistant from the light source of the same module. In other words, the spacing between a light source (i.e., a distal end portion of a light source optical conduit) and the detectors (i.e., distal end portions of optical conduits for each detector) are maintained at the same fixed distance on each module to ensure homogeneous coverage over specific areas and to facilitate processing of the detected signals. The fixed spacing also provides consistent spatial (lateral and depth) resolution across the target area of interest, e.g., brain tissue. Moreover, maintaining a known distance between the light source, e.g., light emitter, and the detector allows subsequent processing of the detected signals to infer spatial (e.g., depth localization, inverse modeling) information about the detected signals. Detectors of a module may be alternatively disposed on the module as may serve a particular implementation.

In some examples, modular assembly 400 can conform to a three-dimensional (3D) surface of the human subject's head, maintain tight contact of the detectors with the human subject's head to prevent detection of ambient light, and maintain uniform and fixed spacing between light sources and detectors. The wearable module assemblies may also accommodate a large variety of head sizes, from a young child's head size to an adult head size, and may accommodate a variety of head shapes and underlying cortical morphologies through the conformability and scalability of the wearable module assemblies. These exemplary modular assemblies and systems are described in more detail in U.S. patent application Ser. Nos. 17/176,470; 17/176,487; 17/176,539; 17/176,560; 17/176,460; and Ser. No. 17/176,466, which applications have been previously incorporated herein by reference in their respective entireties.

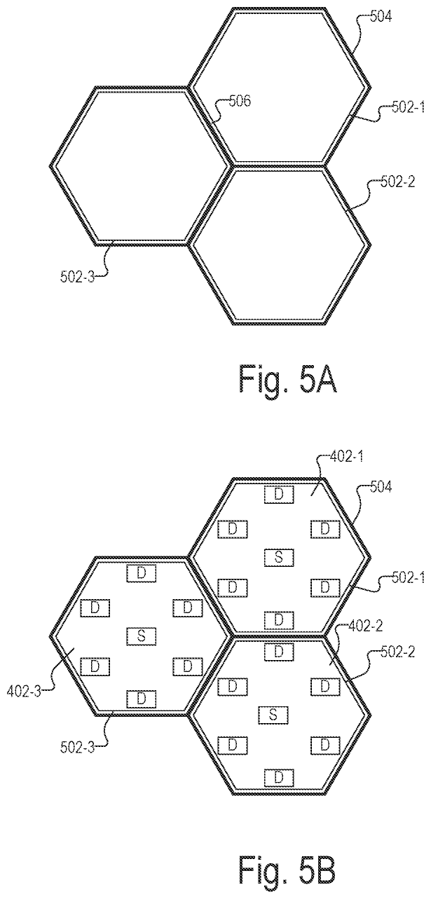

In FIG. 4, modules 402 are shown to be adjacent to and touching one another. Modules 402 may alternatively be spaced apart from one another. For example, FIGS. 5A-5B show an exemplary implementation of modular assembly 400 in which modules 402 are configured to be inserted into individual slots 502 (e.g., slots 502-1 through 502-3, also referred to as cutouts) of a wearable assembly 504. In particular, FIG. 5A shows the individual slots 502 of the wearable assembly 504 before modules 402 have been inserted into respective slots 502, and FIG. 5B shows wearable assembly 504 with individual modules 402 inserted into respective individual slots 502.

Wearable assembly 504 may implement wearable assembly 302 and may be configured as headgear and/or any other type of device configured to be worn by a user.

As shown in FIG. 5A, each slot 502 is surrounded by a wall (e.g., wall 506) such that when modules 402 are inserted into their respective individual slots 502, the walls physically separate modules 402 one from another. In alternative embodiments, a module (e.g., module 402-1) may be in at least partial physical contact with a neighboring module (e.g., module 402-2).

Each of the modules described herein may be inserted into appropriately shaped slots or cutouts of a wearable assembly, as described in connection with FIGS. 5A-5B. However, for ease of explanation, such wearable assemblies are not shown in the figures.

As shown in FIGS. 4 and 5B, modules 402 may have a hexagonal shape. Modules 402 may alternatively have any other suitable geometry (e.g., in the shape of a pentagon, octagon, square, rectangular, circular, triangular, free-form, etc.).

As another example, brain interface system 102 may be implemented by a wearable multimodal measurement system configured to perform both optical-based brain data acquisition operations and electrical-based brain data acquisition operations, such as any of the wearable multimodal measurement systems described in U.S. Patent Application Publication Nos. 2021/0259638 and 2021/0259614, which publications are incorporated herein by reference in their respective entireties.

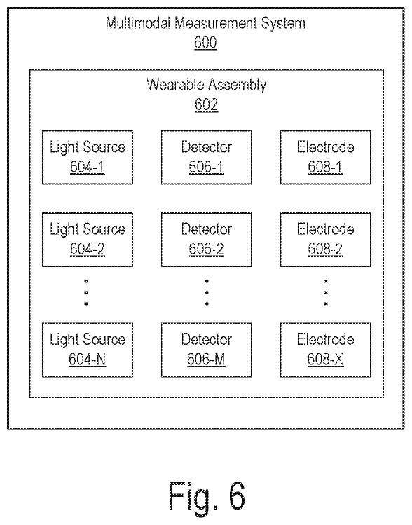

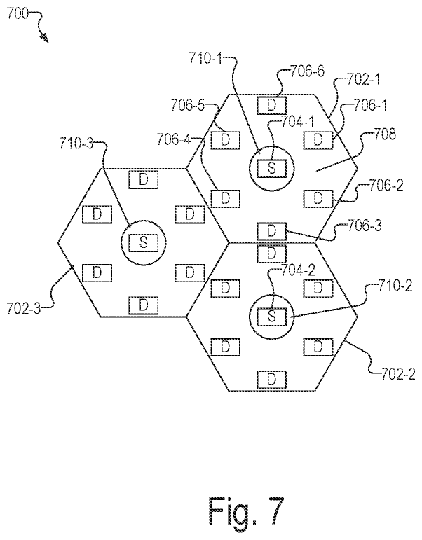

To illustrate, FIGS. 6-7 show various multimodal measurement systems that may implement brain interface system 102. The multimodal measurement systems described herein are merely illustrative of the many different multimodal-based brain interface systems that may be used in accordance with the systems and methods described herein.

FIG. 6 shows an exemplary multimodal measurement system 600 in accordance with the principles described herein. Multimodal measurement system 600 may at least partially implement optical measurement system 200 and, as shown, includes a wearable assembly 602 (which is similar to wearable assembly 302), which includes N light sources 604 (e.g., light sources 604-1 through 604-N, which are similar to light sources 304), M detectors 606 (e.g., detectors 606-1 through 606-M, which are similar to detectors 306), and X electrodes (e.g., electrodes 608-1 through 608-X). Multimodal measurement system 600 may include any of the other components of optical measurement system 200 as may serve a particular implementation. N, M, and X may each be any suitable value (i.e., there may be any number of light sources 604, any number of detectors 606, and any number of electrodes 608 included in multimodal measurement system 600 as may serve a particular implementation).

Electrodes 608 may be configured to detect electrical activity within a target (e.g., the brain). Such electrical activity may include electroencephalogram (EEG) activity and/or any other suitable type of electrical activity as may serve a particular implementation. In some examples, electrodes 608 are all conductively coupled to one another to create a single channel that may be used to detect electrical activity. Alternatively, at least one electrode included in electrodes 608 is conductively isolated from a remaining number of electrodes included in electrodes 608 to create at least two channels that may be used to detect electrical activity.

FIG. 7 shows an illustrative modular assembly 700 that may implement multimodal measurement system 600. As shown, modular assembly 700 includes a plurality of modules 702 (e.g., modules 702-1 through 702-3). While three modules 702 are shown to be included in modular assembly 700, in alternative configurations, any number of modules 702 (e.g., a single module up to sixteen or more modules) may be included in modular assembly 700. Moreover, while each module 702 has a hexagonal shape, modules 702 may alternatively have any other suitable geometry (e.g., in the shape of a pentagon, octagon, square, rectangular, circular, triangular, free-form, etc.).

Each module 702 includes a light source (e.g., light source 704-1 of module 702-1 and light source 704-2 of module 702-2) and a plurality of detectors (e.g., detectors 706-1 through 706-6 of module 702-1). In the particular implementation shown in FIG. 7, each module 702 includes a single light source and six detectors. Alternatively, each module 702 may have any other number of light sources (e.g., two light sources) and any other number of detectors. The various components of modular assembly 700 shown in FIG. 7 are similar to those described in connection with FIG. 4.

As shown, modular assembly 700 further includes a plurality of electrodes 710 (e.g., electrodes 710-1 through 710-3), which may implement electrodes 608. Electrodes 710 may be located at any suitable location that allows electrodes 710 to be in physical contact with a surface (e.g., the scalp and/or skin) of a body of a user. For example, in modular assembly 700, each electrode 710 is on a module surface configured to face a surface of a user's body when modular assembly 700 is worn by the user. To illustrate, electrode 710-1 is on surface 708 of module 702-1. Moreover, in modular assembly 700, electrodes 710 are located in a center region of each module 702 and surround each module's light source 704. Alternative locations and configurations for electrodes 710 are possible.

As another example, brain interface system 102 may be implemented by a wearable magnetic field measurement system configured to perform magnetic field-based brain data acquisition operations, such as any of the magnetic field measurement systems described in U.S. patent application Ser. No. 16/862,879, filed Apr. 30, 2020 and published as US20200348368A1; U.S. Provisional Application No. 63/170,892, filed Apr. 5, 2021, U.S. patent application Ser. No. 17/338,429, filed Jun. 3, 2021, and Ethan J. Pratt, et al., “Kernel Flux: A Whole-Head 432-Magnetometer Optically-Pumped Magnetoencephalography (OP-MEG) System for Brain Activity Imaging During Natural Human Experiences,” SPIE Photonics West Conference (Mar. 6, 2021), which applications and publications are incorporated herein by reference in their entirety. In some examples, any of the magnetic field measurement systems described herein may be used in a magnetically shielded environment which allows for natural user movement as described for example in U.S. Provisional Patent Application No. 63/076,015, filed Sep. 9, 2020, and U.S. patent application Ser. No. 17/328,235, filed May 24, 2021 and published as US2021/0369166A1, which applications are incorporated herein by reference in their entirety.

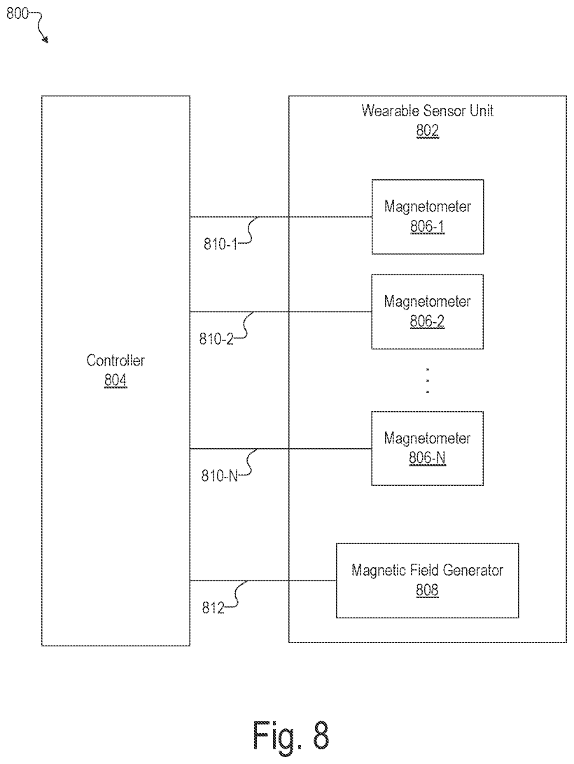

FIG. 8 shows an exemplary magnetic field measurement system 800 (“system 800”) that may implement brain interface system 102. As shown, system 800 includes a wearable sensor unit 802 and a controller 804. Wearable sensor unit 802 includes a plurality of magnetometers 806-1 through 806-N (collectively “magnetometers 806”, also referred to as optically pumped magnetometer (OPM) modular assemblies as described below) and a magnetic field generator 808. Wearable sensor unit 802 may include additional components (e.g., one or more magnetic field sensors, position sensors, orientation sensors, accelerometers, image recorders, detectors, etc.) as may serve a particular implementation. System 800 may be used in magnetoencephalography (MEG) and/or any other application that measures relatively weak magnetic fields.

Wearable sensor unit 802 is configured to be worn by a user (e.g., on a head of the user). In some examples, wearable sensor unit 802 is portable. In other words, wearable sensor unit 802 may be small and light enough to be easily carried by a user and/or worn by the user while the user moves around and/or otherwise performs daily activities, or may be worn in a magnetically shielded environment which allows for natural user movement as described more fully in U.S. Provisional Patent Application No. 63/076,015, and U.S. patent application Ser. No. 17/328,235, filed May 24, 2021 and published as US202110369166A1, previously incorporated by reference.

Any suitable number of magnetometers 806 may be included in wearable sensor unit 802. For example, wearable sensor unit 802 may include an array of nine, sixteen, twenty-five, or any other suitable plurality of magnetometers 806 as may serve a particular implementation.

Magnetometers 806 may each be implemented by any suitable combination of components configured to be sensitive enough to detect a relatively weak magnetic field (e.g., magnetic fields that come from the brain). For example, each magnetometer may include a light source, a vapor cell such as an alkali metal vapor cell (the terms “cell”, “gas cell”, “vapor cell”, and “vapor gas cell” are used interchangeably herein), a heater for the vapor cell, and a photodetector (e.g., a signal photodiode). Examples of suitable light sources include, but are not limited to, a diode laser (such as a vertical-cavity surface-emitting laser (VCSEL), distributed Bragg reflector laser (DBR), or distributed feedback laser (DFB)), light-emitting diode (LED), lamp, or any other suitable light source. In some embodiments, the light source may include two light sources: a pump light source and a probe light source.

Magnetic field generator 808 may be implemented by one or more components configured to generate one or more compensation magnetic fields that actively shield magnetometers 806 (including respective vapor cells) from ambient background magnetic fields (e.g., the Earth's magnetic field, magnetic fields generated by nearby magnetic objects such as passing vehicles, electrical devices and/or other field generators within an environment of magnetometers 806, and/or magnetic fields generated by other external sources). For example, magnetic field generator 808 may include one or more coils configured to generate compensation magnetic fields in the Z direction, X direction, and/or Y direction (all directions are with respect to one or more planes within which the magnetic field generator 808 is located). The compensation magnetic fields are configured to cancel out, or substantially reduce, ambient background magnetic fields in a magnetic field sensing region with minimal spatial variability.

Controller 804 is configured to interface with (e.g., control an operation of, receive signals from, etc.) magnetometers 806 and the magnetic field generator 808. Controller 804 may also interface with other components that may be included in wearable sensor unit 802.

In some examples, controller 804 is referred to herein as a “single” controller 804. This means that only one controller is used to interface with all of the components of wearable sensor unit 802. For example, controller 804 may be the only controller that interfaces with magnetometers 806 and magnetic field generator 808. It will be recognized, however, that any number of controllers may interface with components of magnetic field measurement system 800 as may suit a particular implementation.

As shown, controller 804 may be communicatively coupled to each of magnetometers 806 and magnetic field generator 808. For example, FIG. 8 shows that controller 804 is communicatively coupled to magnetometer 806-1 by way of communication link 810-1, to magnetometer 806-2 by way of communication link 810-2, to magnetometer 806-N by way of communication link 810-N, and to magnetic field generator 808 by way of communication link 812. In this configuration, controller 804 may interface with magnetometers 806 by way of communication links 810-1 through 810-N (collectively “communication links 810”) and with magnetic field generator 808 by way of communication link 812.

Communication links 810 and communication link 812 may be implemented by any suitable wired connection as may serve a particular implementation. For example, communication links 810 may be implemented by one or more twisted pair cables while communication link 812 may be implemented by one or more coaxial cables. Alternatively, communication links 810 and communication link 812 may both be implemented by one or more twisted pair cables. In some examples; the twisted pair cables may be unshielded.

Controller 804 may be implemented in any suitable manner. For example, controller 804 may be implemented by a field-programmable gate array (FPGA), an application specific integrated circuit (ASIC), a digital signal processor (DSP), a microcontroller, and/or other suitable circuit together with various control circuitry.

In some examples, controller 804 is implemented on one or more printed circuit boards (PCBs) included in a single housing. In cases where controller 804 is implemented on a PCB, the PCB may include various connection interfaces configured to facilitate communication links 810 and 812. For example, the PCB may include one or more twisted pair cable connection interfaces to which one or more twisted pair cables may be connected (e.g., plugged into) and/or one or more coaxial cable connection interfaces to which one or more coaxial cables may be connected (e.g., plugged into).

In some examples, controller 804 may be implemented by or within a computing device.

In some examples, a wearable magnetic field measurement system may include a plurality of optically pumped magnetometer (OPM) modular assemblies, which OPM modular assemblies are enclosed within a housing sized to fit into a headgear (e.g., brain interface system 102) for placement on a head of a user (e.g.; human subject). The OPM modular assembly is designed to enclose the elements of the OPM optics, vapor cell, and detectors in a compact arrangement that can be positioned close to the head of the human subject. The headgear may include an adjustment mechanism used for adjusting the headgear to conform with the human subject's head. These exemplary OPM modular assemblies and systems are described in more detail in U.S. Provisional Patent Application No. 63/170,892, filed Apr. 5, 2021, and U.S. patent application Ser. No. 17/338,429, filed Jun. 3, 2021, previously incorporated by reference.

At least some of the elements of the OPM modular assemblies, systems which can employ the OPM modular assemblies, and methods of making and using the OPM modular assemblies have been disclosed in U.S. Patent Application Publications Nos. 2020/0072916; 2020/0056263; 2020/0025844; 2020/0057116; 2019/0391213; 2020/0088811; 2020/0057115; 2020/0109481; 2020/0123416; 2020/0191883; 2020/0241094; 2020/0256929; 2020/030987 2020/0334559; 2020/0341081; 2020/0381128; 2020/0400763; 2021/0011094; 2021/0015385; 2021/0041512; 2021/0041513; 2021/0063510; and 20210139742, and U.S. Provisional Patent Application Ser. Nos. 62/689,696; 62/699,596; 62/719,471; 62/719,475; 62/719,928; 62/723,933; 62/732,327; 62/732,791; 62/741,777; 62/743,343; 62/747,924; 62/745,144; 62/752,067; 62/776,895; 62/781,418; 62/796,958; 62/798,209; 62/798,330; 62/804,539; 62/826,045; 62/827,390; 62/836,421; 62/837,574; 62/837,587; 62/842,818; 62/855,820; 62/858,636; 62/860,001; 62/865,049; 62/873,694; 62/874,887; 62/883,399; 62/883,406; 62/888,858; 62/895,197; 62/896,929; 62/898,461; 62/910,248; 62/913,000; 62/926,032; 62/926,043; 62/933,085; 62/960,548; 62/971,132; 63/031,469; 63/052,327; 63/076,015; 63/076,880; 63/080,248; 63/135,364; 63/136,415; and 63/170,892, all of which are incorporated herein by reference in their entireties.

In some examples, one or more components of brain interface system 102, FIG. 1, (e.g., one or more computing devices) may be configured to be located off the head of the user.

In each of the different brain interface system implementations described herein, the sleep routine data may be based on the type of operations performed by the different brain interface system implementations. For example, if brain interface system 102 is implemented by an optical measurement system configured to perform optical-based brain data acquisition operations, the brain activity data may be based on the optical-based brain data acquisition operations. As another example, if brain interface system 102 is implemented by a multimodal measurement system configured to perform optical-based brain data acquisition operations and electrical-based brain data acquisition operations, the brain activity data may be based on the optical-based brain data acquisition operations and the electrical-based brain data acquisition operations. As another example, if brain interface system 102 is implemented by a magnetic field measurement system configured to perform magnetic field-based brain data acquisition operations, the brain activity data may be based on the magnetic field-based brain data acquisition operations.

Returning to FIG. 1, sleep tracking device 104 may have any suitable form factor. For example, sleep tracking device 104 may be implemented by a wrist wearable device, a chest strap, an armband wearable device, a ring wearable on a finger, an ankle band, etc. In some examples, sleep tracking device 104 may implement a time domain-based optical measurement system configured to non-invasively measure blood oxygen saturation (SaO2) through Time-Resolved Pulse Oximetry (TR-SpO2), such as one or more of the devices described in more detail in U.S. Provisional Patent Application No. 63/134,479, filed Jan. 6, 2021, U.S. Provisional Patent Application No. 63/154,116, filed Feb. 26, 2021, U.S. Provisional Patent Application No. 63/160,995, filed Mar. 15, 2021, and U.S. Provisional Patent Application No. 63/179,080, filed Apr. 23, 2021, which applications are incorporated herein by reference.

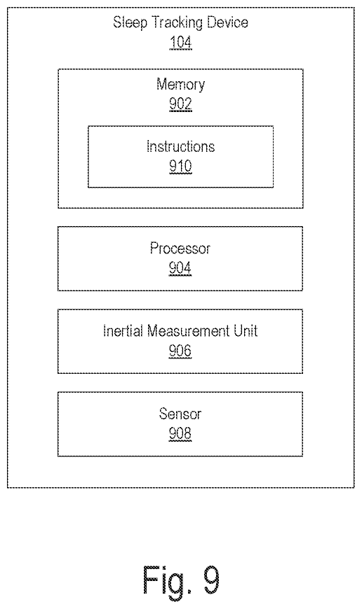

FIG. 9 illustrates various components that may be included in sleep tracking device 104. As shown, sleep tracking device 104 may include memory 902, a processor 904, an inertial measurement unit (IMU) 906, and a sensor 908. Additional or alternative components may be included in sleep tracking device 104 as may serve a particular implementation.

Memory 902 may maintain (e.g., store) executable data used by processor 904 to perform one or more of the operations described herein as being performed by sleep tracking device 104. For example, memory 902 may store instructions 910 that may be executed by processor 904 to generate sleep tracking data. Instructions 910 may be implemented by any suitable application, program, software, code, and/or other executable data instance. Memory 902 may also maintain any data received, generated, managed, used, and/or transmitted by processor 904.

Processor 904 may be configured to perform (e.g., execute instructions 910 stored in memory 902 to perform) various operations described herein as being performed by sleep tracking device 104. Examples of such operations are described herein.

IMU 906 may be detect movement of the user (e.g., while the user is sleeping or trying to go to sleep). IMU 906 may have any suitable number of axes (e.g., up to nine axes, such as three accelerometer axes, three gyroscope axes, and three magnetometer axes).

Sensor 908 may be implemented by one or more sensors configured to sense various types of sensor input. For example, sensor 908 may be implemented by a body temperature sensor configured to detect a temperature of a body of the user, a skin conductivity sensor configured to detect a conductivity of skin of the user, an ambient light sensor configured to track light exposure in an environment of the user, and/or a microphone configured to detect sound (e.g., disturbances and snoring while the user sleeps).

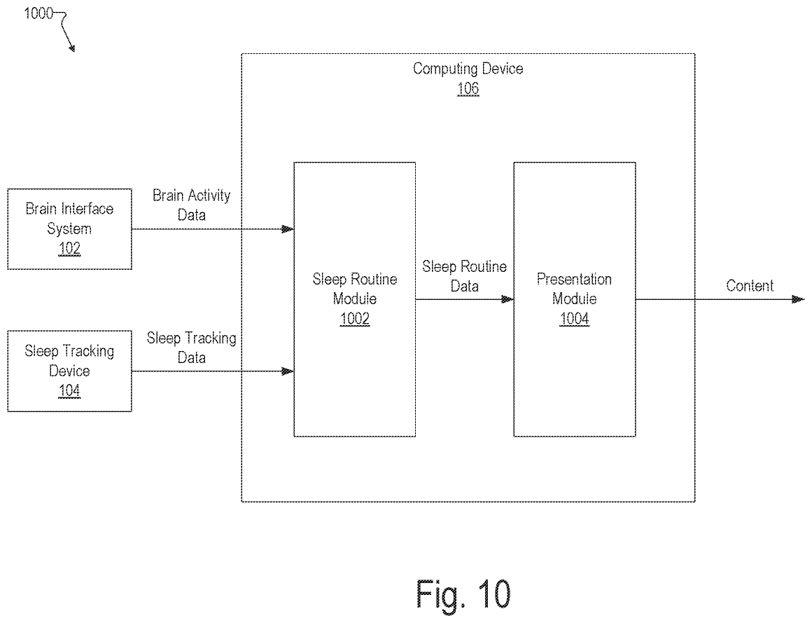

Various implementations of computing device 106, FIG. 1, generating sleep routine data and performing one or more operations based on the sleep routine data will now be described in connection with FIGS. 10-15.

FIG. 10 illustrates a configuration 1000 in which computing device 106 is configured to present content associated with the target sleep routine to the user. As shown, computing device 106 may include a sleep routine module 1002 and a presentation module 1004, each of which may be implemented by any suitable combination of hardware and/or software.

Sleep routine module 1002 may be configured to generate sleep routine data based on brain activity data output by brain interface system 102 and sleep tracking data output by sleep tracking device 104. Exemplary manners in which sleep routine module 1002 (i.e., computing device 106) may generate sleep routine data based on brain activity data and sleep tracking data will now be described.

In some examples, sleep routine module 1002 may be configured to generate sleep routine data by determining an effect of one or more attributes of a user's sleep during a sleeping time period (e.g., a night) on how well the user is able to function (e.g., mentally) during an awake time period (e.g., day-time hours) during which the user is awake following the sleeping time period.

To illustrate, using the sleep tracking data, sleep routine module 1002 may determine certain attributes of a user's sleep during a particular night. For example, using the sleep tracking data, sleep routine module 1002 may determine that the user went to bed at a certain time and woke up at a certain time, that the user took a certain number of minutes to fall asleep, that the user had a certain number of minutes of REM sleep, that the user's heart rate varied by a certain amount while in different stages of sleep, that the room in which the user slept was at a certain temperature, etc.

Furthermore, using the brain activity data, sleep routine module 1002 may determine how well the user is able to function (e.g., mentally) during the day that follows the particular night. For example, using brain activity data, sleep routine module 1002 may determine how well the user is able to focus on certain tasks during the day, how well the user is able to exercise impulse control when presented with various temptations and/or choices throughout the day, what the user's mental state is throughout the day (e.g., how stressed and/or happy the user is throughout the day), how well the user gets along with others throughout the day, etc.

Sleep routine module 1002 may then correlate the sleep tracking data for the particular night with the brain activity data for the day that follows the night to determine how the various attributes of the user's sleep may have influenced how well the user was able to function during the following day. Such correlation may be performed in any suitable manner.

For example, sleep routine module 1002 may obtain a sleep performance score and a brain activity score for the user. These scores may be obtained in any suitable manner. For example, sleep routine module 1002 may generate the sleep performance score based on the sleep tracking data output by sleep tracking device 104. Alternatively, the sleep performance score may be included in the sleep tracking data output by sleep tracking device 104, such that sleep routine module 1002 obtains the sleep performance score by receiving the sleep tracking data. Likewise, sleep routine module 1002 may generate the brain activity score based on the brain activity data output by brain interface system 102. Alternatively, the brain activity score may be included in the brain activity data output by brain interface system 102, such that sleep routine module 1002 obtains the brain activity score by receiving the brain activity data.

Sleep routine module 1002 may then correlate the sleep performance score with the brain activity score to determine how the sleep performance score affects the brain activity score. The correlation may be implemented using any suitable statistical analysis, machine learning model, and/or other type of processing algorithm as may serve a particular implementation. Based on the correlation, sleep routine module 1002 may generate the sleep routine data.

For example, based on the correlation, sleep routine module 1002 may determine that one or more characteristics of the user's sleep had a positive effect on the user's ability to function. In this case, the sleep routine data generated by sleep routine module 1002 may indicate that these one or more characteristics should stay unchanged for future periods of sleep. To illustrate, based on the correlation, sleep routine module 1002 may determine that the temperature of the room in which the user slept had a positive effect on the user's quality of sleep and, consequently, the user's ability to function the next day. Based on this, sleep routine module 1002 may generate sleep routine data that indicates that the temperature of the room should remain unchanged during subsequent periods of sleep for the user.

As another example, sleep routine module 1002 may determine that one or more characteristics of the user's sleep may have negatively impacted the user's ability to function and that the one or more characteristics should be adjusted for subsequent periods of sleep. To illustrate, based on the correlation, sleep routine module 1002 may determine that the user went to bed at a time that caused the user to not function as well as other days where the user had gone to bed at a different time. Based on this, sleep routine module 1002 may generate sleep routine data that indicates that the user should go to bed at a different time for subsequent periods of sleep.

Additionally or alternatively, sleep routine module 1002 may be configured to generate sleep routine data by determining an effect brain activity data recorded while the user is awake has on the quality level of sleep that the user obtains during a subsequent period of sleep.

For example, sleep routine module 1002 may obtain a brain activity score for the user during a particular time period of being awake (e.g., day-time hours) and a sleep performance score for a time period of sleep (e.g., a night) following the time period of being awake. These scores may be obtained in any of the ways described herein.

Sleep routine module 1002 may then correlate the brain activity score with the sleep performance score to determine how the brain activity score affects the sleep performance score. The correlation may be implemented using any suitable statistical analysis, machine learning model, and/or other type of processing algorithm as may serve a particular implementation. Based on the correlation, sleep routine module 1002 may generate the sleep routine data.

For example, based on the correlation, sleep routine module 1002 may determine that one or more activities performed by the user have a positive affect on the quality of sleep obtained by the user. In this case, the sleep routine data generated by sleep routine module 1002 may indicate that these one or more activities should stay unchanged for future sleep routines. To illustrate, based on the correlation, sleep routine module 1002 may determine that the user not eating after a certain time in the evening results in a relatively high sleep quality level for the user. Based on this, sleep routine module 1002 may generate sleep routine data that indicates that the user should continue to not eat after this time each day.

As another example, based on the correlation, sleep routine module 1002 may determine that one or more activities performed by the user have a negative affect on the quality of sleep obtained by the user. In this case, the sleep routine data generated by sleep routine module 1002 may indicate that these one or more activities should be changed for future sleep routines. To illustrate, based on the correlation, sleep routine module 1002 may determine that the user listening to a certain type of music prior to going to bed has a negative affect on the quality of sleep obtained by the user. Based on this, sleep routine module 1002 may generate sleep routine data that indicates that the user should avoid listening to this type of music within a certain amount of time of going to bed.

While the above examples involve generating and correlating sleep performance scores and brain activity scores, it will be recognized that sleep routine module 1002 may additionally or alternatively be configured to generate sleep tracking data based on sleep routine data and brain activity data in any other suitable manner.

Presentation module 1004 may be configured to generate content based on the sleep routine data generated by sleep routine module 1002. The content may include any suitable information associated with the sleep routine data that may be presented to the user. For example, the content may include information that summarizes the target sleep routine (e.g., that lists a number of actions that the user should take throughout the day to adhere to the target sleep routine), a score indicative of how well the user adheres to the target sleep routine, a reminder to perform a task associated with the target sleep routine, a suggestion to adjust one or more settings of a device (e.g., a temperature setting of a heating and/or cooling device, a color tone or intensity of a light, a noise level of a noise machine, etc.), and/or any other type of content as may serve a particular implementation.

Presentation module 1004 may present the content in any suitable manner. For example, presentation module 1004 may visually and/or audibly present the content, for example, by way of a graphical user interface and/or a speaker implemented by computing device 106. As another example, presentation module 1004 may present the content by directing a display device and/or an audio device not included in computing device 106 to present the content. This may be performed in any suitable manner. In some examples, the content is presented by way of an application executed by a mobile device (e.g., a mobile device used by the user).

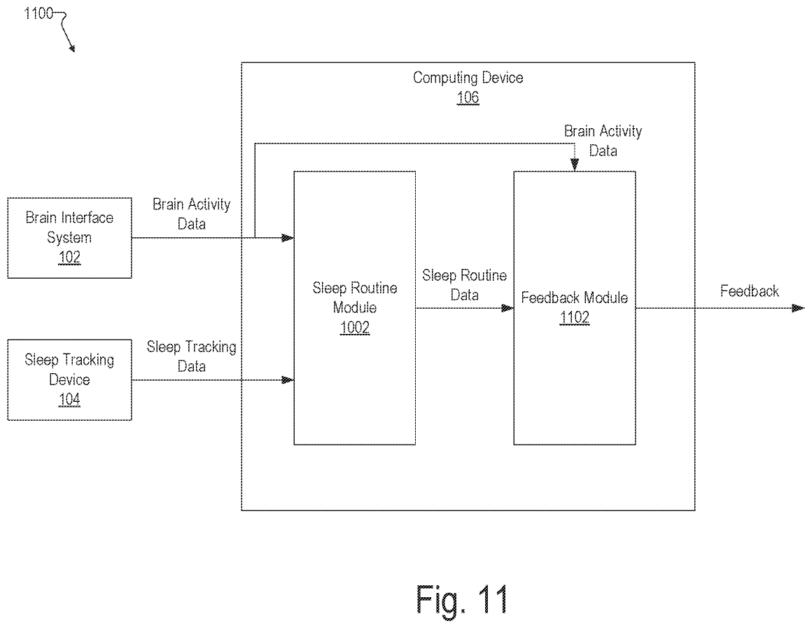

FIG. 11 illustrates a configuration 1100 in which computing device 106 is additionally or alternatively configured to assist the user in adhering to the target sleep routine by providing feedback to the user.

As shown, in configuration 1100, computing device 106 includes sleep routine module 1002, which generates sleep routine data as described herein. Computing device 106 further includes a feedback module 1102 configured to receive the sleep routine data and the brain activity data as inputs. Based on the brain activity data (which may, in this example, be provided in substantially real-time as the brain activity data is being generated), feedback module 1102 may determine that the user is being presented with a choice that affects the target sleep routine and provide feedback configured to assist the user in making the choice. The feedback may include one or more alerts, electrical stimulation, auditory stimulation, tactile feedback, and/or any other type of feedback as may serve a particular implementation.

For example, the brain activity data may indicate that the user is being tempted to eat something at a particular time of day that would negatively impact the user's target sleep routine. Based on this, feedback module 1102 may provide feedback (e.g., a visual and/or audible alert, electrical stimulation, auditory stimulation, etc.) that assists the user in withstanding the temptation to eat (e.g., by reminding the user that eating would negatively impact the user's target sleep routine).

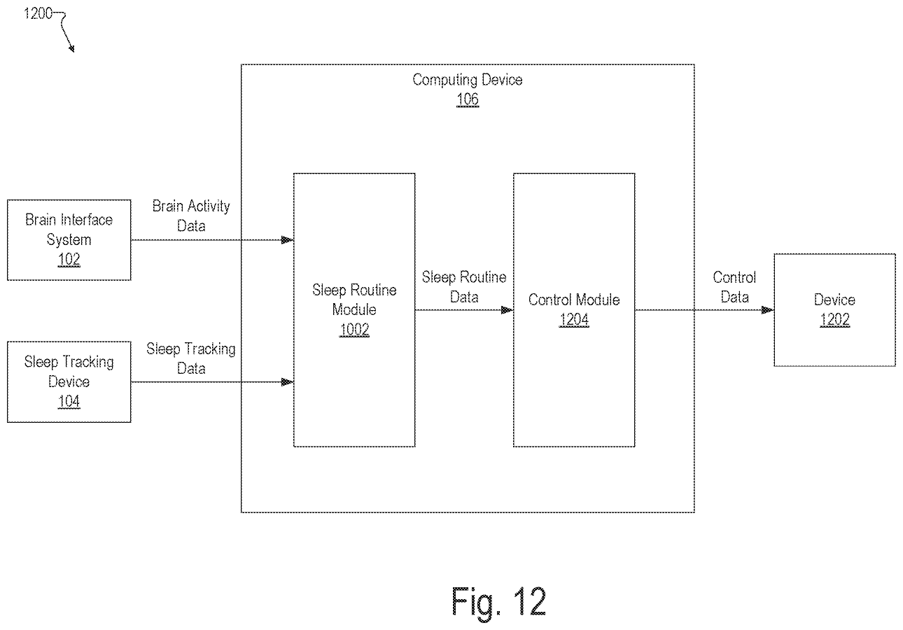

FIG. 12 illustrates a configuration 1200 in which computing device 106 is additionally or alternatively configured to control one or more settings of a device 1202 separate from computing device 106 to assist the user in adhering to the target sleep routine. Device 1202 may be any controllable device, such as a heating and/or cooling device (e.g., a furnace, an air conditioner, an electric blanket or pad, etc.), a light source (e.g., an overhead light, a lamp, etc.), a media player (e.g., a music player, a television, a gaming device, etc.), a mobile device (e.g., a mobile phone, a tablet computer, etc.), a sound machine (e.g., a white noise machine, etc.), and/or any other device that has one or more settings that may be controlled by computing device 106.

As shown, in configuration 1200, computing device 106 includes sleep routine module 1002, which generates sleep routine data as described herein. Computing device 106 further includes a control module 1204 configured to control a setting of device 1202 based on the sleep routine data.

For example, control module 1204 may be configured to transmit control data to device 1202, where the control data may include any suitable data configured to control one or more settings of device 1202. The control data may be transmitted to device 1202 in any suitable manner (e.g., wirelessly by way of a network, via a wired connection, etc.).

To illustrate, device 1202 may be implemented by a heating device, such as a heating blanket, a heating pad, a furnace, and/or any other device configured to provide heat. In this example, the control data output by control module 1204 may be configured to adjust a temperature of the heating device to a value specified by the sleep routine data.

As another example, device 1202 may be implemented by a cooling device, such as an air conditioning unit and/or any other device configured to provide cooling for the user. In this example, the control data output by control module 1204 may be configured to adjust a temperature of the cooling device to a value specified by the sleep routine data.

As another example, device 1202 may be implemented by a light source, such as an overhead light, a lamp, and/or any other device configured to provide light. In this example, the control data output by control module 1204 may be configured to adjust a property (e.g., a brightness level, a color, a hue, etc.) of the light output by the light source to a value specified by the sleep routine data.

As another example, device 1202 may be implemented by a media player, such as a television, a computing device, a gaming device, a music player, and/or any other device configured to present visual and/or audio content. In this example, the control data output by control module 1204 may be configured to adjust a presentation setting (e.g., a volume level, a brightness level, an on/off state, a particular type of media content that is being presented, etc.) of the media player to a value specified by the sleep routine data.

As another example, device 1202 may be implemented by a mobile device, such as a mobile phone, a tablet computer, a mobile gaming device, a mobile music player, and/or any other device configured to be portable and usable by the user. In this example, the control data output by control module 1204 may be configured to adjust a setting (e.g., a volume level, a brightness level, an on/off state, a particular type of media content that is being presented, etc.) of the mobile device to a value specified by the sleep routine data.

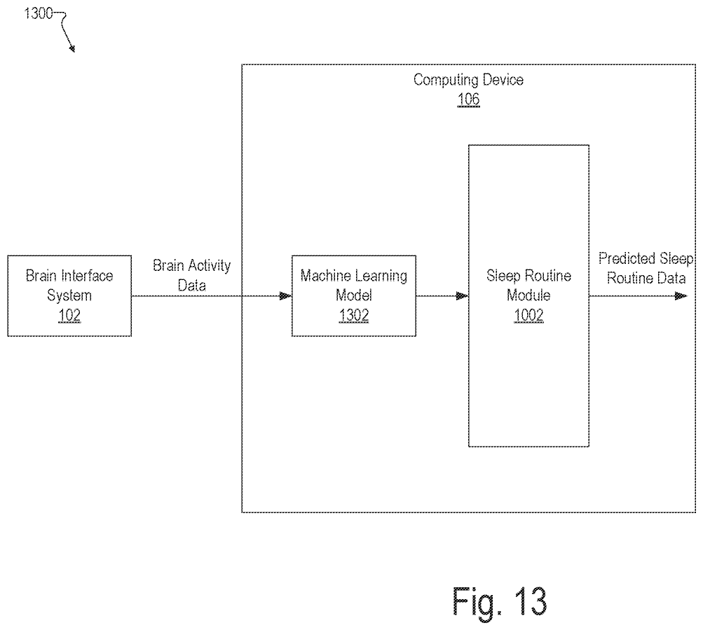

FIG. 13 illustrates a configuration 1300 in which computing device 106 is configured to apply the brain activity data to a machine learning model 1302. Based on an output of the machine learning model 1302, sleep routine module 1002 may generate predicted sleep routine data representative of a predicted target sleep routine for the user. This predicted sleep routine data may be generated without using sleep tracking data, and may, in some instances, be used as a baseline from which the sleep routine data representative of the actual target sleep routine for the user is generated. In some examples, configuration 1300 may be used when the user does not have access to a sleep tracking device.

Machine learning model 1302 may be supervised and/or unsupervised as may serve a particular implementation and may be configured to implement one or more decision tree learning algorithms, association rule learning algorithms, artificial neural network learning algorithms, deep learning algorithms, bitmap algorithms, and/or any other suitable data analysis technique as may serve a particular implementation.

In some examples, machine learning model 1302 is implemented by one or more neural networks, such as one or more deep convolutional neural networks (CNN) using internal memories of its respective kernels (filters), recurrent neural networks (RNN), and/or long/short term memory neural networks (LSTM). Machine learning model 1302 may be multi-layer. For example, machine learning model 1302 may be implemented by a neural network that includes an input layer, one or more hidden layers, and an output layer.

Data representative of machine learning model 1302 may be stored within computing device 106, as shown in FIG. 13. Additionally or alternatively, machine learning model 1302 may be maintained by one or more computing devices remote from computing device 106 (e.g., one or more computing devices communicatively coupled to computing device 106 by way of a network).

In some examples, machine learning model 1302 is trained using sleep routine data for a plurality of users. In this manner, machine learning model 1302 may be configured to predict a target sleep routine for the user based on brain activity data (and not sleep tracking data).

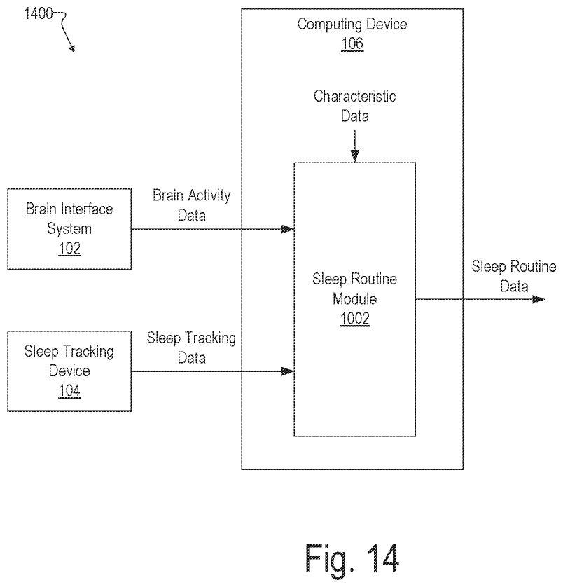

FIG. 14 illustrates a configuration 1400 in which sleep routine module 1002 is configured to receive characteristic data representative of a desired characteristic for the user. The characteristic data may be received in the form of user input, for example. Sleep routine module 1002 may use the characteristic data, together with the brain activity data and the sleep tracking data, to generate the sleep routine data.

The desired characteristic represented by the characteristic data may be any mental or emotional characteristic that the user desires to possess. For example, the desired characteristic may include one or more physiological brain states and/or mental brain states, e.g., joy, excitement, relaxation, contentment, confidence, calmness, focus, attention, impulse control, creativity, a positive attitude, etc. Sleep routine module 1002 may accordingly adjust the target sleep routine to assist the user in achieving the desired characteristic.

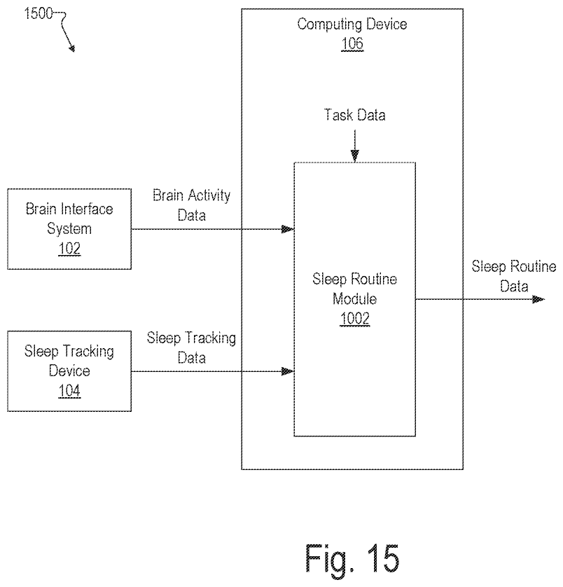

FIG. 15 illustrates a configuration 1500 in which sleep routine module 1002 is configured to receive task data representative of an upcoming task that the user is to perform. The task data may be received in the form of user input, for example. Sleep routine module 1002 may use the task data, together with the brain activity data and the sleep tracking data, to generate the sleep routine data.

For example, the user may provide user input indicating that the user has to take a test in a certain subject at a certain time of day. Based on this user input, sleep routine module 1002 may adjust the target sleep routine to assist the user in performing well on the test.

One or more of the configurations shown in FIGS. 10-15 may be combined such that computing device 106 may be configured to perform any of the operations described in connection with FIGS. 10-15.

In some examples, sleep routine module 1002 may modify the sleep routine data over time as one or more of additional brain activity data is output by brain interface system 102 or additional sleep tracking data is output by sleep tracking device 104. This may be performed in any suitable manner.

In some examples, sleep routine module 1002 may be configured to synchronize the brain activity data with the sleep tracking data. For example, the brain activity data may include first timestamp data and the sleep tracking data may include second timestamp data. Sleep routine module 1002 may to synchronize the brain activity data with the sleep tracking data based on the first and second timestamp data in any suitable manner.

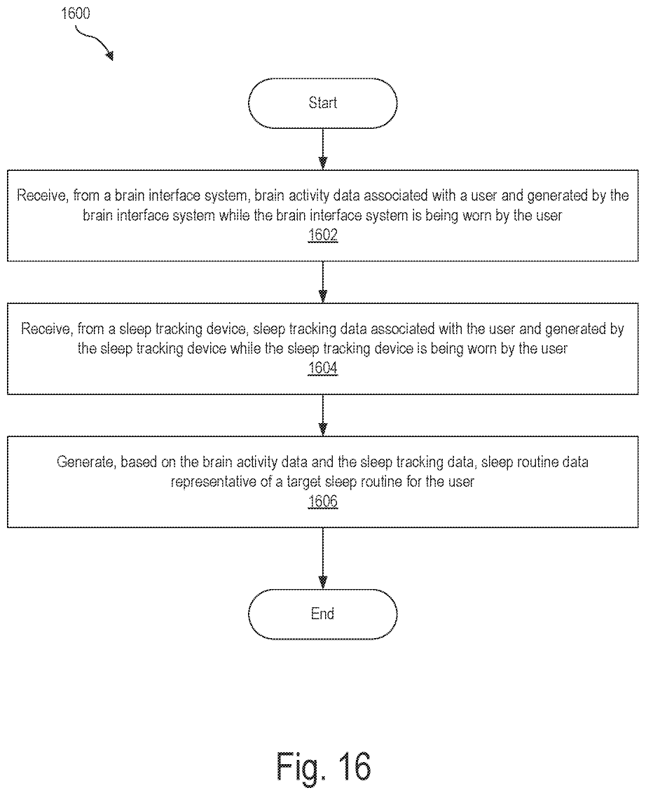

FIG. 16 illustrates an exemplary method 1600. While FIG. 16 illustrates exemplary operations according to one embodiment, other embodiments may omit, add to, reorder, and/or modify any of the operations shown in FIG. 16. One or more of the operations shown in FIG. 16 may be performed by computing device 106 and/or any implementation thereof. Each of the operations illustrated in FIG. 16 may be performed in any suitable manner.

At operation 1602, a computing device receives, from a brain interface system, brain activity data associated with a user and generated by the brain interface system while the brain interface system is being worn by the user.

At operation 1604, the computing device receives, from a sleep tracking device, sleep tracking data associated with the user and generated by the sleep tracking device while the sleep tracking device is being worn by the user.

At operation 1606, the computing device generates, based on the brain activity data and the sleep tracking data, sleep routine data representative of a target sleep routine for the user.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Exemplary non-volatile storage media include, but are not limited to, read-only memory, flash memory, a solid-state drive, a magnetic storage device (e.g. a hard disk, a floppy disk, magnetic tape, etc.), ferroelectric random-access memory (“RAM”), and an optical disc (e.g., a compact disc, a digital video disc, a Blu-ray disc, etc.). Exemplary volatile storage media include, but are not limited to, RAM (e.g., dynamic RAM).

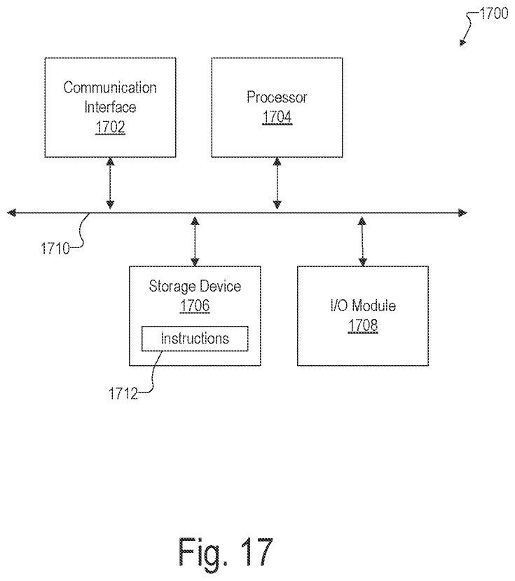

FIG. 17 illustrates an exemplary computing device 1700 that may be specifically configured to perform one or more of the processes described herein. Any of the systems, units, computing devices, and/or other components described herein may be implemented by computing device 1700.

As shown in FIG. 17, computing device 1700 may include a communication interface 1702, a processor 1704, a storage device 1706, and an input/output (“I/O”) module 1708 communicatively connected one to another via a communication infrastructure 1710. While an exemplary computing device 1700 is shown in FIG. 17, the components illustrated in FIG. 17 are not intended to be limiting. Additional or alternative components may be used in other embodiments. Components of computing device 1700 shown in FIG. 17 will now be described in additional detail.

Communication interface 1702 may be configured to communicate with one or more computing devices. Examples of communication interface 1702 include, without limitation, a wired network interface (such as a network interface card), a wireless network interface (such as a wireless network interface card), a modem, an audio/video connection, and any other suitable interface.

Processor 1704 generally represents any type or form of processing unit capable of processing data and/or interpreting, executing, and/or directing execution of one or more of the instructions, processes, and/or operations described herein. Processor 1704 may perform operations by executing computer-executable instructions 1712 (e.g., an application, software, code, and/or other executable data instance) stored in storage device 1706.

Storage device 1706 may include one or more data storage media, devices, or configurations and may employ any type, form, and combination of data storage media and/or device. For example, storage device 1706 may include, but is not limited to, any combination of the non-volatile media and/or volatile media described herein. Electronic data, including data described herein, may be temporarily and/or permanently stored in storage device 1706. For example, data representative of computer-executable instructions 1712 configured to direct processor 1704 to perform any of the operations described herein may be stored within storage device 1706. In some examples, data may be arranged in one or more databases residing within storage device 1706.

I/O module 1708 may include one or more I/O modules configured to receive user input and provide user output. I/O module 1708 may include any hardware, firmware, software, or combination thereof supportive of input and output capabilities. For example, I/O module 1708 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1708 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1708 is configured to provide graphical data to a display for presentation to a user. The graphical data may be representative of one or more graphical user interfaces and/or any other graphical content as may serve a particular implementation.

An illustrative system includes a brain interface system configured to be worn by a user and to output brain activity data associated with the user; a sleep tracking device configured to be worn by the user and to output sleep tracking data associated with the user; and a computing device configured to generate, based on the brain activity data and the sleep tracking data, sleep routine data representative of a target sleep routine for the user.

Another illustrative system includes a memory storing instructions and a processor communicatively coupled to the memory and configured to execute the instructions to: receive, from a brain interface system, brain activity data associated with a user and generated by the brain interface system while the brain interface system is being worn by the user; receive, from a sleep tracking device, sleep tracking data associated with the user and generated by the sleep tracking device while the sleep tracking device is being worn by the user; and generate, based on the brain activity data and the sleep tracking data, sleep routine data representative of a target sleep routine for the user.

An illustrative method includes receiving, by a computing device from a brain interface system, brain activity data associated with a user and generated by the brain interface system while the brain interface system is being worn by the user; receiving, by the computing device from a sleep tracking device, sleep tracking data associated with the user and generated by the sleep tracking device while the sleep tracking device is being worn by the user; and generating, by the computing device based on the brain activity data and the sleep tracking data, sleep routine data representative of a target sleep routine for the user.

An illustrative non-transitory computer-readable medium may store instructions that, when executed, direct a processor of a computing device to: receive, from a brain interface system, brain activity data associated with a user and generated by the brain interface system while the brain interface system is being worn by the user; receive, from a sleep tracking device, sleep tracking data associated with the user and generated by the sleep tracking device while the sleep tracking device is being worn by the user; and generate, based on the brain activity data and the sleep tracking data, sleep routine data representative of a target sleep routine for the user.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. A system comprising:
a brain interface system configured to be worn by a user and to output brain activity data associated with the user;
a sleep tracking device configured to be worn by the user and to output sleep tracking data associated with the user; and
a computing device configured to generate, based on the brain activity data and the sleep tracking data, sleep routine data representative of a target sleep routine for the user.

2. The system of claim 1, wherein the brain interface system comprises an optical measurement system configured to perform optical-based brain data acquisition operations, the brain activity data based on the optical-based brain data acquisition operations.

3. The system of claim 2, wherein the optical measurement system comprises:
a wearable assembly configured to be worn by the user and comprising:
a plurality of light sources each configured to emit light directed at a brain of the user, and
a plurality of detectors configured to detect arrival times for photons of the light after the light is scattered by the brain, the brain activity data based on the arrival times.

4. The system of claim 3, wherein the detectors each comprise a plurality of single-photon avalanche diode (SPAD) circuits.

5. The system of claim 3, wherein the wearable assembly further comprises:
a first module comprising a first light source included in the plurality of light sources and a first set of detectors included in the plurality of detectors; and
a second module physically distinct from the first module and comprising a second light source included in the plurality of light sources and a second set of detectors included in the plurality of detectors.

6. The system of claim 5, wherein the first and second modules are configured to be removably attached to the wearable assembly.

7. The system of claim 1, wherein the brain interface system comprises a multimodal measurement system configured to perform optical-based brain data acquisition operations and electrical-based brain data acquisition operations, the brain activity data based on the optical-based brain data acquisition operations and the electrical-based brain data acquisition operations.

8. The system of claim 7, wherein the multimodal measurement system comprises:
a wearable assembly configured to be worn by the user and comprising:
a plurality of light sources each configured to emit light directed at a brain of the user,
a plurality of detectors configured to detect arrival times for photons of the light after the light is scattered by the brain, and
a plurality of electrodes configured to be external to the user and detect electrical activity of the brain, the brain activity based on the arrival times and the electrical activity.

9. The system of claim 8, wherein the wearable assembly further comprises:
a first module comprising a first light source included in the plurality of light sources and a first set of detectors included in the plurality of detectors; and
a second module physically distinct from the first module and comprising a second light source included in the plurality of light sources and a second set of detectors included in the plurality of detectors.

10. The system of claim 9, wherein the plurality of electrodes comprises a first electrode on a surface of the first module and a second electrode on a surface of the second module.

11. The system of claim 10, wherein the first electrode surrounds the first light source on the surface of the first module.

12. The system of claim 1, wherein the sleep tracking device comprises one or more of a wrist wearable device, a chest strap, an armband wearable device, or a ring wearable on a finger of the user.

13. The system of claim 1, wherein the sleep tracking device comprises a time domain-based optical measurement system configured to non-invasively measure blood oxygen saturation (SaO2) through Time-Resolved Pulse Oximetry (TR-SpO2), the sleep tracking data based on the measured SaO2.

14. The system of claim 1, wherein:
the sleep tracking device comprises one or more of an inertial measurement unit configured to detect movement of the user, a temperature sensor configured to detect a temperature of a body of the user, a skin conductivity sensor configured to detect a conductivity of skin of the user, an ambient light sensor configured to track light exposure in an environment of the user, or a microphone configured to detect sound; and
the sleep tracking data is based on one or more of the detected movement, the detected temperature, the detected conductivity of the skin, the tracked light exposure, or the detected sound.

15. The system of claim 1, wherein the computing device is further configured to perform an operation based on the sleep routine data.

16. The system of claim 15, wherein the performing the operation comprises presenting content associated with the target sleep routine.

17. The system of claim 16, wherein the content comprises one or more of information that summarizes the target sleep routine, a score indicative of how well the user adheres to the target sleep routine, a reminder to perform a task associated with the target sleep routine, or a suggestion to adjust a setting of a device.

18. The system of claim 15, wherein the performing the operation comprises:
detecting, based on the brain activity data, that the user is presented with a choice that affects the target sleep routine; and
providing, in response to the detecting that the user is presented with the choice, feedback configured to assist the user in making the choice.

19. The system of claim 18, wherein the feedback comprises one or more of an alert, electrical stimulation, or auditory stimulation.

20. The system of claim 15, wherein the performing the operation comprises controlling a setting of a device separate from the computing device.

21. The system of claim 20, wherein the controlling the setting of the device comprises one or more of adjusting a temperature of a heating device, adjusting a temperature of a cooling device, adjusting a property of light provided by a light source, adjusting a presentation setting of a media player, or adjusting a setting of a mobile device.

22. The system of claim 1, wherein the computing device is further configured to:
provide, prior to generating the sleep routine data, the brain activity data to a machine learning model; and
generate, based on an output of the machine learning model, predicted sleep routine data representative of a predicted target sleep routine for the user.

23. The system of claim 22, wherein the machine learning model is trained using sleep routine data for a plurality of users.

24. The system of claim 1, wherein the generating of the sleep routine data comprises:
obtaining a sleep performance score for the user, the sleep performance score based on the sleep tracking data;
obtaining a brain activity score for the user, the brain activity score based on the brain activity data;
correlating the sleep performance score with the brain activity score to determine how the sleep performance score affects the brain activity score; and
generating, based on the correlating, the sleep routine data.

25. The system of claim 1, wherein the generating of the sleep routine data comprises:
obtaining a brain activity score for the user, the brain activity score based on the brain activity data;
obtaining a sleep performance score for the user, the sleep performance score based on the sleep tracking data;
correlating the brain activity score with the sleep performance score to determine how the brain activity score affects the sleep performance score; and
generating, based on the correlating, the sleep routine data.

26. The system of claim 1, wherein:
the computing device is further configured to receive characteristic data representative of a desired characteristic for the user; and
the generating of the sleep routine data is further based on the characteristic data.

27. The system of claim 1, wherein:
the computing device is further configured to receive task data representative of an upcoming task that the user is to perform; and
the generating of the sleep routine data is further based on the task data.

28. The system of claim 1, wherein the computing device is further configured to modify the sleep routine data over time as one or more of additional brain activity data is output by the brain interface system or additional sleep tracking data is output by the sleep tracking device.

29. The system of claim 1, wherein:
the brain activity data comprises first timestamp data;
the sleep tracking data comprises second timestamp data; and
the computing device is configured to synchronize the brain activity data with the sleep tracking data based on the first and second timestamp data.

30. The system of claim 1, wherein the computing device is configured to be worn by the user at a same time that the brain interface system and the sleep tracking device are being worn by the user.

31-75. (canceled)
